(19) United States
(12) Patent Application Publication
(10) Pub. No.: US 2017/0221339 A1
(43) Pub. Date: Aug. 3, 2017

(54) UV Reference Indicator for Estimating Webbing Tensile Strength

(71) Applicant: Robert E. Golz, Swansea, MA (US)

(72) Inventor: Robert E. Golz, Swansea, MA (US)

(21) Appl. No.: 15/491,900

(22) Filed: Apr. 19, 2017

Related U.S. Application Data
(63) Continuation-in-part of application No. 14/280,890, filed on May 19, 2014, now abandoned.

(51) Int. Cl. G08B 21/18; G01L 5/04; G01N 33/36

(52) U.S. Cl. CPC G08B 21/182; G01N 33/367; G01L 5/047

(57) ABSTRACT

In general, the present invention has to do with a UV reference indicator calibrated to represent the color or shade of a UV reactive dye after a predetermined exposure time to a UV radiation source.

### CROSS-REFERENCE TO RELATED APPLICATIONS

This application is a continuation in part of:
1. U.S. patent application Ser. No. 14/280,890, entitled “A Webbing System Incorporating a UV Reactive Dye and Corresponding UV Reference Indicator”, naming Robert E. Golz as inventor, filed May 19, 2014.

### BACKGROUND

1. Field of Use

These teachings relate generally to a webbing or lanyards incorporating a Ultra Violet (UV) reactive dye and corresponding UV reference indicator

2. Description of Prior Art (Background)

The history of protecting workers at heights in the infant stages of fall protection was as basic as tying one end of a rope around the workers safety belt and the other to an anchor point. Over the years the technology became more sophisticated and formalized.

By the seventies most workers at heights were required by their employees to wear safety belts. However, many workers were negligent about securing the safety belts at a tie off point. The eighties required 100% tie off where the worker was required to use two lanyards attached to his safety belt. Used properly the worker would be secured with one lanyard 100% of the time. During the early nineties safety belts gave way to safety harnesses with a “D” ring on the back side of the harness to avoid serious back injuries and finally full body harnesses.

After years in development, the American National Committee on Standards for Fall Protection in 1992 issued the ANSI Z359.1 fall protection standard, later revised in 1999. This standard addressed the technological advances made by manufacturers of fall protection equipment. The Z359.1 is a voluntary compliance standard for the four elements of Personal Fall Arrest Systems. Included were the anchorage's, body wear, connectors and deceleration devices.

The state of the art today for fall arrest systems is the use of Self Retracting Lifelines for fall restraint. The system is comprised of narrow webbing wound on to a reel that has a locking mechanism, similar to automotive locking retractor systems. The locking mechanism limits the fall distance to 24 inches, rather than the older technology which uses a personal energy absorber to limit the free fall distance to six feet.

Currently the American National Committee on Standards for Fall Protection is developing an expanded family of related standards. When completed the new standards will be composed of 18 separate standards to address every aspect of Fall Protection. Europe (EN 360:2002), Canada (CAN/CSA Z259.2.1-98 (R201) and Australia (AS/NZS 1891.1; 2007) each have Fall Protection standards similar to the Z359 family of standards.

Each of the Z359 standards addresses a specific issue in Fall Protection. One of which is the Self Retracting Lifeline. This standard requires webbing used in the Self Retracting Lifelines meet a dynamic performance test for retractable type fall arrestor in horizontal use. There is a clause in the proposed Z359.14 document, for a Self-Retracting Lanyard with Leading Edge Capability (SRL-LE). “A self-retracting device suitable for applications where during use the device in not necessarily mounted or anchored overhead and may be at foot level and where the possible free fall is up to 5 ft. (1.5 m) that includes integral means to withstand impact loading of the line constituent with a sharp or abrasive edge during fall arrest and for controlling fall arrest forces on the user.

Webbing's that have been used for Self-Retracting Lifelines traditionally have been made using nylon or polyester fibers with dimensions that were between ½″ wide up to 1½″ wide and thickness' of between 0.050″ and 0.100″.

When working at heights, it is standard industry practice (often guided by legal requirements of governmental agencies such as OSHA) to employ some sort of fall protection safety device. As noted earlier, for such devices to effectively protect users from dangerous falls, the device webbing must be of sufficient strength to bring a fall to a stop and to then hold the user above the ground. Thus, the structural integrity and/or strength of such devices may be critical in preventing serious bodily injury from a fall. Currently, there is no known method to quickly, and objectively, determine if fall protection safety devices that have been used over some period of time still retain their original structural integrity/strength characteristics, e.g., webbing or lanyards, or if they have been significantly degraded to the point where they should be retired from service. At best, some industries may simply apply a uniform rule (based mainly on estimation) that devices should be retired a certain number of years after manufacture.

However, this a very rough estimate, and does not take into account the specific use and/or environmental exposures of a particular device. For example, a device using webbing or lanyards may have been stored away in inventory for many years, such that its actual useful lifespan might be many years more than the uniform rule would assume. This could lead to costly waste as useful devices are needlessly discarded. On the other hand, a particular device might be used in an environment where it is exposed to one or more harmful substances that could be detrimental to its structural integrity/strength. This could lead to a device being used beyond its actual useful lifespan (based on the application of a uniform rule).

Various attempts have been made to incorporate end of service life indicators into safety harnesses, belts, and lanyards. For example the patent application publication US20130056302 discloses various methods of combing UV sensing devices, known in the art, with webs and lanyards. For example, a UV sensing device in said publication may “turn color” when exposed to a sufficient amount of UV light. However, this approach fails to remove the subjective analysis of a color change. In other words, the publication fails to address how much of a color change is required for a determination that the webbing has been exposed to an excessive amount of UV radiation. The publication also fails to disclose or suggest how to address the person-to-person subjective color perception question in that the color perceived by person A is often not the exact same color perceived by person B.

Similarly, patent application publication US20120171918A1 discloses a tracing yarn that changes color after exposure heat, flame, and long term excessive UV light and the like. This publication also fails to address how much of a color change is required for a determination that the webbing has been exposed to an excessive amount of UV radiation. In other words, there is no objective standard by which to correlate the color change to structural integrity/strength characteristics of a lanyard or webbing.

In addition, the traditional webbing made of nylon and or polyester has not been shown to meet the Sharp edge testing requirements. Various methods of weaving and combinations of high performance fibers, e.g., high tenacity yarns have been made to attempt meet the requirements of the Leading Edge Testing; but, have failed the standards requirements for sharp edge testing.

In other industrial applications medium or heavy weight industrial webbing is widely used, for instance, for truckload restraint systems, container tie downs, and other relatively high strength applications. Such webbings are typically woven in a double or multiple weave construction having an upper layer of fabric and a lower layer of fabric. The two or more layers are, of course, joined by many binders and further preferably include an inner layer of stuffer yarns.

Typically, medium or heavy weight industrial webbing is woven from synthetic multifilament yarn. While polypropylene is used when high strength is not necessary, high strength applications typically use nylon (polyamid) or polyester. Both nylon and polyester yarns have very high tenacity. Nylon yarn, however, because of its superior elongation actually requires more work to break. Polyester, because it has less elongation is beneficial since its elongation under load is less.

Therefore it is an objective of this invention to provide webbing wherein the remaining life expectancy of the webbing may be objectively determined by visual inspection.

With these and other objects in view, as will be apparent to those skilled in the art, the invention resides in the combination of parts set forth in the specification and covered in the claims appended hereto.

### BRIEF SUMMARY

The foregoing and other problems are overcome, and other advantages are realized, in accordance with the presently preferred embodiments of these teachings.

The invention is be directed towards one or more end of service life indicators operable to indicate exposure to one or more degrading conditions; wherein the one or more end of service life indicators change in visual appearance when exposed to sufficient concentration and duration of degrading condition to weaken the strength of the webbing.

The invention is also directed towards an Ultra Violet (UV) reference system for estimating tensile strength of webbing dyed with UV sensitive dye, the system includes at least one non UV sensitive reference indicator comprising a visual characteristic (e.g, color) corresponding to a predetermined UV exposure time of the UV sensitive dye. The predetermined UV exposure time corresponds to a tensile strength of the webbing.

In accordance with one embodiment of the invention an Ultra Violet (UV) reference system for estimating tensile strength of webbing dyed with non UV sensitive dye is provided, The system includes at least one reference indicator comprising UV sensitive dye; and at least one non UV sensitive reference indicator, the at least one non comprising a visual characteristic (e.g., color) corresponding to a predetermined UV exposure time of the UV sensitive dye, wherein the predetermined UV exposure time corresponds to a tensile strength of the webbing.

The invention is also directed towards webbing, or strands of webbing, dyed with a UV reactive dye which changes color and/or shading corresponding to UV exposure and one or more corresponding non-UV sensitive reference indicators incorporated within the webbing, wherein the reference indicators are correlated to a strength loss or end of service life. The one or more non UV sensitive reference indicators may be calibrated to tensile strengths such that the UV exposed dye may be visually matched with the reference indicator to objectively determine the webbing's remaining service life.

Another aspect of the invention is directed towards a UV reference indicator card wherein the card contains at least one UV reference indicator (e.g., color) to be matched with the webbing or thread dyed with a UV reactive dye which changes color and/or shading corresponding to UV exposure. The UV references may be deposited on said card by any suitable means such as UV resistant inks or pigments.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1, in accordance with the present invention, is a pictorial illustration of UV sensitive dyed webbing exposed to UV radiation over time and incorporating a calibrated non-UV sensitive reference indicator indicating when the UV webbing has lost a predetermined percentage of its original tensile strength;

FIG. 2, in accordance with the present invention, is a pictorial illustration of non UV sensitive webbing incorporating a UV sensitive yarn or thread and a calibrated non-UV sensitive reference indicator indicating when the UV webbing has lost a predetermined percentage of its original tensile strength;

FIG. 3 is a graph of tensile strength loss over time of the web shown in FIG. 1; and

FIG. 4 is a pictorial illustration of a tensile strength loss reference card in accordance with the present invention.

### DETAILED DESCRIPTION

The following brief definition of terms shall apply throughout the application:

The term “outer” or “outside” refers to a direction away from a user, while the term “inner” or “inside” refers to a direction towards a user;

The term “comprising” means including but not limited to, and should be interpreted in the manner it is typically used in the patent context;

The phrases “in one embodiment,” “according to one embodiment,” and the like generally mean that the particular feature, structure, or characteristic following the phrase may be included in at least one embodiment of the present invention, and may be included in more than one embodiment of the present invention (importantly, such phrases do not necessarily refer to the same embodiment);

If the specification describes something as “exemplary” or an “example,” it should be understood that refers to a non-exclusive example; and

If the specification states a component or feature “may,” “can,” “could,” “should,” “preferably,” “possibly,” “typically,” “optionally,” “for example,” or “might” (or other such language) be included or have a characteristic, that particular component or feature is not required to be included or to have the characteristic.

Disclosed embodiments may relate to fall protection safety devices having one or more end of service life indicators. Typically, the devices are fall arresting personal safety devices, such as safety harnesses, belts, lanyards, anchor slates, lifelines (which may be retractable), and the like, by way of nonexclusive example. Embodiments of such devices often might comprise webbing of sufficient structural integrity, strength, and/or tenacity to effectively protect against dangerous falls (by, for example, catching and/or holding the user in the event the user should fall from a height), along with one or more end of service life indicators to allow a user to quickly and easily inspect the safety of the device and to determine whether the device is still effective for safety purposes or whether there is sufficient risk of degradation that the device should be retired.

Persons of skill will be familiar with the types of webbing typically used for such devices. By way of example, the webbing typically may comprise nylon, polyester, and/or combinations thereof. In some embodiments, the webbing might typically include only a single type of structural support fiber/material. While other manufacturing methods may be appropriate, in embodiments such webbing may typically be woven. The dimensions (such as width and thickness of the webbing material), number of layers of material and stitching patterns and/or needle-punching used in creating the webbing might typically be selected so that the webbing has sufficient structural integrity/strength/tenacity for safety purposes (for example, to catch and support the weight of a user falling from a height). Oftentimes, the minimum strength requirement for webbing in such devices is legally set (based for example on OSHA requirements) and/or is set by industry standard or custom. So, for example, the minimum legal standard might be 5,000 pounds (supportable by the webbing), while in another industry the standard or custom might be to have a minimum strength of 7,000 pounds (supportable by the webbing). Regardless, the webbing must possess sufficient strength to provide the safety restraint or fall arresting capabilities for embodiments of the device.

Referring to FIG. 11 there is shown, in accordance with the present invention, a pictorial illustration of UV dyed webbing exposed to UV radiation over time and incorporating a reference indicator 121 indicating when the UV webbing has lost, in this example, approximately 20% of its original tensile strength. It is understood that webbing 12A through 12F represents one piece of webbing exposed to UV radiation over time. It will further be understood that for purposes of this description, it is desired that the webbing, dyed with UV sensitive indicator dye, be replaced when the webbing has lost 20% of its original tensile strength. It will also be understood that the reference indicator may be incorporated in any suitable position within the webbing. For example, the reference indicator 121 may be a one ply plain weave webbing incorporating dyed red, or any suitable color, yarns that approximately match the color shade exhibited by the UV indicator dye after a predetermined number of UV exposure hours that correspond to, for example, a 20% tensile strength reduction of the webbing. The one ply webbing may be sewn onto, or otherwise attached, to the edges of the webbing with the UV indicator dyes, similar to a piping. It will also be appreciated that the reference indicator may be any suitable thread, yarn, or other material.

Webbing 12A is observed at time t₀, or in other words the webbing has not been exposed to UV radiation and there is 0% tensile strength loss. Thus, the color or shade of the 20% loss reference indicator 121 does not match the color or shade of the webbing 12A previously dyed with UV sensitive dye. It will be understood that the reference indicator 12A may be any suitable reference indicator having a suitable visual characteristic, such as, for example, a color. For example the reference indicator 12A may be a light shade of non UV sensitive red previously determined to correspond to a 20% tensile strength loss. It will also be understood that the reference indicator 121 may be any suitable UV resistant reference indicator. The UV sensitive webbing may also be any initial suitable color and/or color shade which will ultimately match the reference indicator 121 after a predetermined amount of UV exposure.

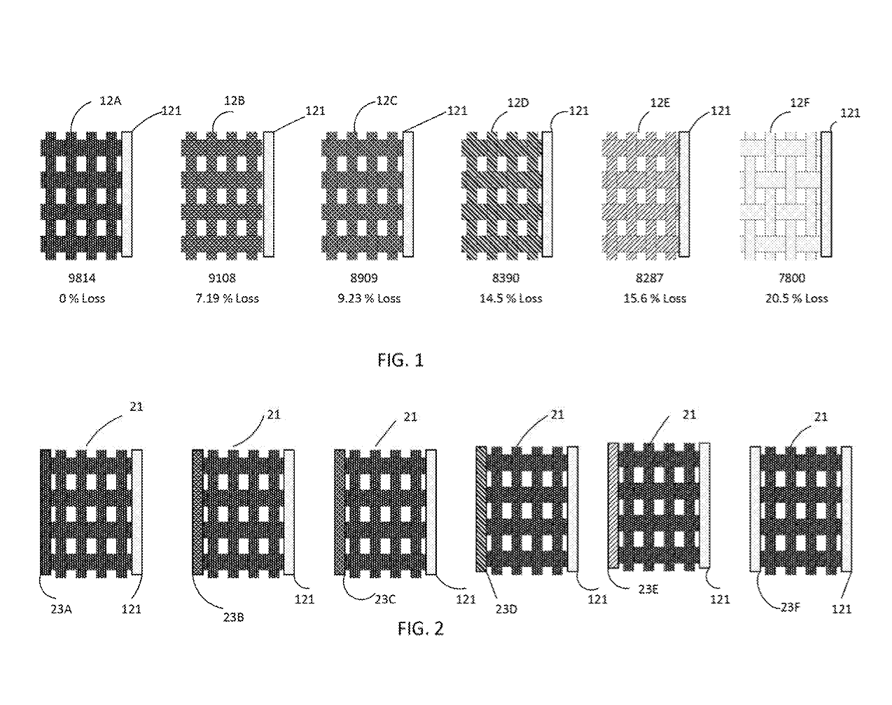

Still referring to FIG. 1, there is illustrated webbing 12B. Webbing 12B is exposed to UV radiation for cumulative amount of time which deteriorates the webbing tensile strength by 7.19%. Again, the webbing color, does not match the calibrated 20% loss reference indictor 121.

Likewise, webbing 12C illustrates a cumulative exposure to UV radiation resulting in 9.23% webbing tensile strength loss. Again, the webbing pattern, or color, does not match the calibrated 20% loss reference indictor 121.

FIG. 1 continues illustrating the tensile strength loss of the webbing due to cumulative UV exposure. At 12F the UV sensitive dyed webbing approximately matches the calibrated 20% non-UV sensitive reference indicator 121; which signifies that the UV dyed webbing has been exposed to UV radiation for a certain number of hours corresponding to a 20% loss of the webbing's original tensile strength.

It will be understood that any suitable number of calibrated reference indicator values may be employed within the webbing shown in FIG. 1. For example, there may be reference indicators corresponding to 5%, 10%, 15%, and/or 20%, or more. Using more than one reference indicator allows for visual determination of a UV dyed webbing current tensile strength loss.

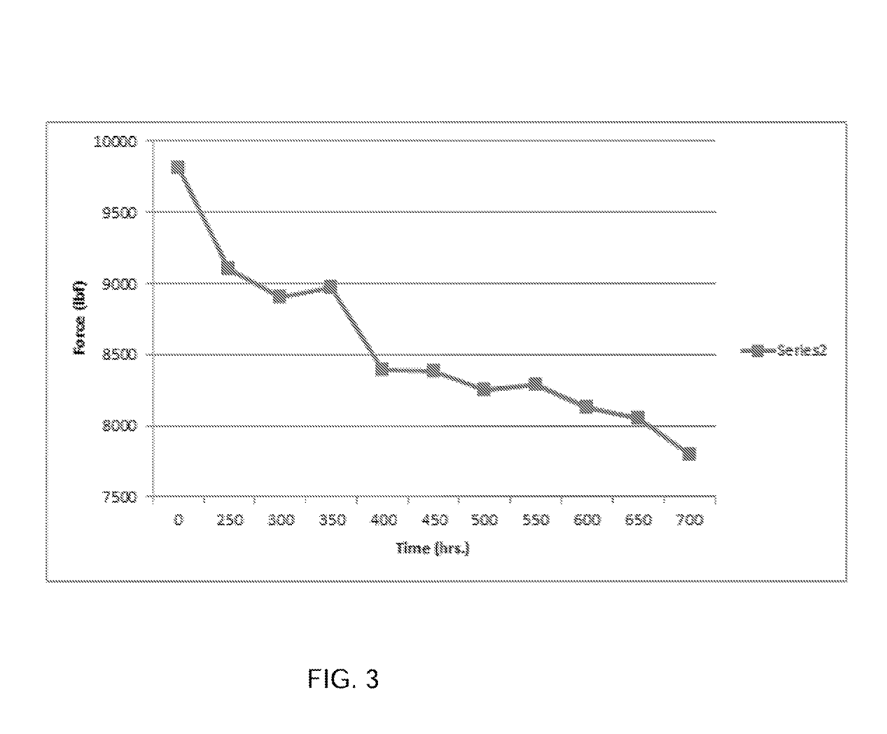

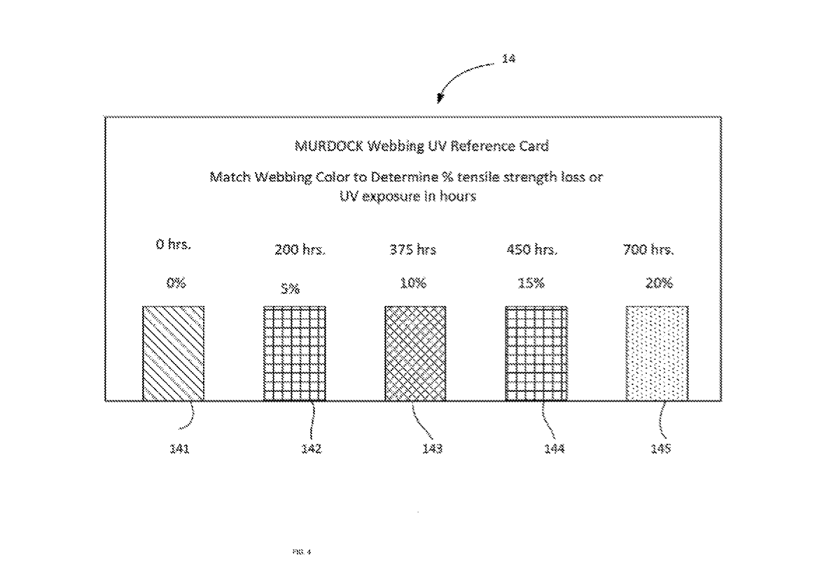

Referring also to FIG. 3 there is shown a graph of tensile strength loss over time of the UV dyed webbing shown in FIG. 1. At time t₀ the webbing's tensile strength, for this example, is at its maximum of approximately 9,800 lbs. After UV exposure of approximately 700 hours, the UV sensitive webbing tensile strength has decreased by approximately 20%, thus indicating, for this example, that the UV dyed webbing has reached its end of live service.

It is understood from FIG. 1, FIG. 2, and FIG. 3 that the UV dye exposure reaction, e.g., changes in coloring or shading, is independent of the webbing material. In other words, the UV dyed webbing will change color and/or shade as a function of time and the UV dye chemical properties. However, the initial tensile strength and subsequent tensile strength loss of the webbing will be dependent upon the webbing material. Thus, for example, UV dyed webbing constructed from any suitable weft, warp, and stuffers yarns such as, for example, nylon or polyester, may have different initial tensile strengths and subsequent tensile strength loss over time than similar webbing which may include steel stuffer yarns, and/or multi-component yarns. Thus, a calibrated 20% loss reference indicator may be a different color, or color shade, for webbing comprising only synthetic materials than a calibrated 20% loss indicator for webbing including multi-component and/or metal wires.

Referring also to FIG. 4 there is shown a pictorial illustration of a tensile strength loss reference card in accordance with the present invention. The loss reference card provides a ready reference for visually determining the current tensile strength loss of UV dyed webbing. For example, UV reference 141 corresponds to a 0% tensile strength loss. A user holding the card next to webbing, such as shown in FIG. 12, item 12A, can readily determine that the webbing has not been exposed to any appreciable UV radiation. Likewise, the user, holding the card near webbing illustrated in FIG. 12, item 12D can visually match the webbing's color or shade to reference 143 and thus determine that the webbing's current tensile strength loss is approximately 10%. Similarly, when the webbing shown in FIG. 12—item 12F matches reference 145, the user can determine that the webbing tensile strength loss is approximately 20%.

Still referring to FIG. 4, it will be understood that the UV reference indicators may be comprised of any suitable ink, paint, pigment, or combination thereof to reasonably represent the color or color shade of a UV dye exposed to a UV radiation source over time. It will also be appreciated that the UV reference indicators units could be stated in UV exposure hours which can then be correlated to a tensile strength loss table for the particular webbing under analysis. For example, UV loss indicator 143 could represent a cumulative UV exposure to a UV source of approximately 375 hours. Using the UV exposure of 375 hours a user can then determine the approximate tensile strength loss for differently constructed UV dyed webbings, e.g., webbing constructed with steel stuffer wires vs. webbing constructed without steel stuffer wires, from a predetermined look up table.

It should be understood that the foregoing description is only illustrative of the invention. Thus, various alternatives and modifications can be devised by those skilled in the art without departing from the invention. Accordingly, the present invention is intended to embrace all such alternatives, modifications and variances that fall within the scope of the appended claims.

Additionally, the section headings used herein are provided for consistency with the suggestions under 37 C.F.R. 1.77 or to otherwise provide organizational cues. These headings shall not limit or characterize the invention(s) set out in any claims that may issue from this disclosure. Specifically and by way of example, although the headings might refer to a “Field,” the claims should not be limited by the language chosen under this heading to describe the so-called field. Further, a description of a technology in the “Background” is not to be construed as an admission that certain technology is prior art to any invention(s) in this disclosure. Neither is the “Summary” to be considered as a limiting characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of the claims shall be considered on their own merits in light of this disclosure, but should not be constrained by the headings set forth herein.

Use of broader terms such as comprises, includes, and having should be understood to provide support for narrower terms such as consisting of, consisting essentially of, and comprised substantially of. Use of the term “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

What is claimed is:

1. An Ultra Violet (UV) reference system for estimating tensile strength of webbing dyed with UV sensitive dye, the system comprising:
at least one non UV sensitive reference indicator, the at least one non UV sensitive reference indicator comprising:
a visual characteristic corresponding to a predetermined UV exposure time of the UV sensitive dye, wherein the predetermined UV exposure time corresponds to a tensile strength of the webbing.

2. The at least one non UV sensitive reference indicator as in claim 1 comprising at least one thread attached to the webbing dyed with the UV sensitive dye.

3. The at least one thread as in claim 2 further comprising a one ply webbing.

4. The system as in claim 1 further comprising:
at least one second non UV sensitive reference indicator, the at least one second non UV sensitive reference indicator comprising:
a second visual characteristic corresponding to a second predetermined UV exposure time of the UV sensitive dye, wherein the second predetermined UV exposure time corresponds to a second tensile strength of the webbing.

5. An Ultra Violet (UV) reference system for estimating tensile strength of webbing dyed with non UV sensitive dye, the system comprising:
at least one reference indicator comprising UV sensitive dye; and
at least one non UV sensitive reference indicator, the at least one non UV sensitive reference indicator comprising:
a visual characteristic corresponding to a predetermined UV exposure time of the UV sensitive dye, wherein the predetermined UV exposure time corresponds to a tensile strength of the webbing.

6. The at least one reference indicator comprising UV sensitive dye as in claim 5 comprising at least one UV sensitive thread attached to the webbing dyed with the non UV sensitive dye.

7. The at least one non UV sensitive reference indicator as in claim 5 comprising at least one non UV sensitive thread attached to the webbing dyed with the non UV sensitive dye.

8. An Ultra Violet (UV) reference device for estimating tensile strength of webbing containing at least one reference indicator dyed with UV sensitive dye, the device comprising:
at least one thread comprising UV sensitive dye, wherein the at least one thread is attached to the webbing; and
a plurality of non UV sensitive reference indicators, a first one of the plurality of non UV sensitive reference indicators comprising:
a first visual characteristic corresponding to a first predetermined UV exposure time of the UV sensitive dye, wherein the first predetermined UV exposure time corresponds to a first tensile strength of the webbing; and
a second one of the plurality of non UV sensitive reference indicators comprising:
a second visual characteristic corresponding to a second predetermined UV exposure time of the UV sensitive dye, wherein the second predetermined UV exposure time corresponds to a second tensile strength of the webbing.
